(19) United States
(12) Patent Application Publication
(10) Pub. No.: US 2021/0090703 A1
(43) Pub. Date: Mar. 25, 2021

(54) SMART HEALTH MANAGEMENT DEVICES AND SYSTEMS

(71) Applicant: CueMed Inc., Redmond, WA (US)

(72) Inventors: Sripriya Komarlingam Natarajan, Redmond, WA (US); Leonard George, Redmond, WA (US)

(21) Appl. No.: 17/018,851

(22) Filed: Sep. 11, 2020

Related U.S. Application Data
(63) Continuation of application No. PCT/US2019/022269, filed on Mar. 14, 2019.
(60) Provisional application No. 62/643,319, filed on Mar. 15, 2018.

(51) Int. Cl. G16H 20/13; G16H 40/67; G06K 7/10; G06K 19/07; G16H 50/20; H04W 4/80; A61J 7/00; A61J 7/04

(52) U.S. Cl. CPC G16H 20/13; G16H 40/67; G06K 7/10297; G06K 19/0723; A61J 2200/30; H04W 4/80; A61J 7/0076; A61J 7/0481; G16H 50/20

(57) ABSTRACT

The present disclosure provides smart health management devices and systems. The smart health management devices and systems may improve the medication adherence of a user. The smart health management device may comprise a body having a plurality of compartments, one or more proximity sensors, an short-range communication interface, and a one or more computer processors. The smart health management device may be controlled by a mobile electronic device of the user through the computer processors.

### CROSS-REFERENCE

This application is a continuation of International Application No. PCT/US2019/22269, filed Mar. 14, 2019, which claims priority to U.S. Provisional Patent Application No. 62/643,319, filed Mar. 15, 2018, which are entirely incorporated herein by reference.

### BACKGROUND

Poor adherence to long-term treatment of chronic diseases, like hypertension, cardiovascular diseases, HIV/AIDS, or depression is an escalating world-wide problem. It has been found that in developed countries, the average rate of adherence among patients suffering from chronic diseases is only 50%, and the rate is even lower in developing countries. For instance, in Gambia, China, and the United States only 27%, 43% and 51%, respectively, of patients adhere to their medication for high blood pressure. Similar patterns have been discovered for other diseases such as depression (40%-70%), asthma (43% for acute treatments and 28% for maintenance), and HIV/AIDS (37%-83%).

Results of the poor adherence include poor clinical outcomes and considerable morbidity and mortality. Approximately 125,000 deaths per year in the United States are due to medication non-adherence and between 33 and 69 percent of medication-related hospital admissions in the U.S. are due to poor adherence. Another undesirable outcome of poor adherence is the increasing cost of health care. The total cost for non-adherence is estimated as $100-300 billion each year, including both direct and indirect costs.

In order to improve medication adherence, various stakeholders adopt different medication adherence interventions and programs. However, certain interventions and programs that are effective for long-term care are exceedingly complex, labor intensive, costly, and loosely addressing patients' needs. Some medication adherence interventions and programs for long-term care may require more convenient care, information, reminders, self-monitoring, reinforcement, counseling, family therapy, psychological therapy, crisis intervention, manual telephone follow-up, and supportive care.

### SUMMARY

Recognized herein is the need for simple, cost-efficient, and user friendly products or methods to improve medication adherence. A smart health management system may enable a subject of chronic diseases to take his/her medications on time, thus helping to improve the subject's medication adherence. It may allow the subject to receive artificial intelligence-driven, voice-enabled, geospatially sensitive insights of his/her medication adherence, and may significantly lower the cost, increase the efficiency, and improve subject's experience. The smart health management system may comprise a smart health management device that drives adherence, awareness and accountability of a subject (e.g., patient) towards improving a health outcome. By taking the subject's goals and health (e.g., disease) condition into account, the smart health management system may influence the subject to take action on the different aspects of the subject's lifestyle and improve the subject's health.

In an aspect, the present disclosure provides a smart health management device, comprising: a body having a plurality of modules, wherein the plurality of modules comprise one or more compartments, wherein the one or more compartments are configured to hold medication for use by a user; one or more proximity sensors configured to detect a presence of the user in proximity to the body; a short-range communication interface configured to detect communication tags attached to the plurality of modules; and one or more computer processors configured to (i) use the one or more proximity sensors to detect the presence of the user in proximity to the body, (ii) use the short-range communication interface to detect the communication tags, thereby detecting the plurality of modules, and (iii) upon detecting the plurality of modules, provide the user with information corresponding to the medication in the one or more compartments when the presence of the user is detected to be in proximity to the body.

In some embodiments, the information alerts the user to take a given medication according to a selected schedule. In some embodiments, a communication tag of the communication tags provides one or more characteristics of a module of the plurality of modules. In some embodiments, the one or more characteristics indicate when the module is removed from the body and when the module is placed in the body. In some embodiments, the one or more proximity sensors detect the presence of the user when a distance between the body and the user is smaller than a predetermined distance. In some embodiments, the predetermined distance is at most about 1 meter. In some embodiments, the predetermined distance is at most about 0.5 meter.

In some embodiments, the one or more computer processors provides the user with the information on a mobile electronic device of the user. In some embodiments, the information is provided on a user interface of the mobile electronic device of the user. In some embodiments, the body further comprises a camera configured to detect a presence of medication in a compartment of the one or more compartments. In some embodiments, the one or more computer processors provides the user with an alert indicating the presence of medication is detected in the compartment after a time period for the user to take the medication. In some embodiments, a compartment of the one or more compartments comprises an indicator, and wherein the indicator indicates that medication is to be taken from the compartment. In some embodiments, the smart health management system further comprises a sorting unit configured to sort the medication into the one or more compartments.

In another aspect, the present disclosure provides a smart health management device, comprising: a body comprising a base and a plurality of modules, wherein a module of the plurality of modules is configured to hold medication for use by a user; a locking unit configured to (i) lock the module to the base and (ii) unlock the module from the base; one or more user identification units configured to identify the user; and one or more computer processors configured to (i) use the one or more user identification units to identify the user, (ii) upon identifying the user, direct the locking unit to unlock the module from the base, thereby permitting the user to remove the module from the base when the module is unlocked from the base.

In some embodiments, the plurality of modules comprises one or more compartments, and wherein the one or more compartments hold the medication for use by the user. In some embodiments, the body further comprises a camera configured to detect a presence of medication in the module. In some embodiments, the one or more computer processors provides the user with an alert indicating the presence of medication is detected in the module after a time period for the user to take the medication. In some embodiments, the module comprises one or more indicators, and wherein the one or more indicators indicate that medication is to be taken from the module. In some embodiments, the module is locked to the base such that a user cannot access the medication in the module. In some embodiments, the one or more identification units identify the user by facial recognition, finger printing, voice recognition, or personal identification number. In some embodiments, the locking unit is configured to unlock a single module at a time. In some embodiments, the smart health management device further comprises a display configured to display information to a user. In some embodiments, the information comprises dosing information, dosing schedule, and/or patient education. In some embodiments, the smart health management device further comprises a speaker configured to provide audio indication of dosing information and/or dosing schedule.

In another aspect, the present disclosure provides a method for providing a medication to a user, comprising: providing a smart health management device comprising (i) a body having a plurality of modules, (ii) one or more proximity sensors that detect a presence of the user in proximity to the body, and (iii) a short-range communication interface that detects communication tags attached to the plurality of modules, wherein the plurality of modules comprise one or more compartments that hold the medication for use by the user; using the one or more proximity sensors to detect a presence of the user in proximity to the body; using the short-range communication interface to detect the communication tags, thereby detecting the plurality of modules; and providing the user with information corresponding to the medication in the one or more compartments when the presence of the user is detected to be in proximity to the body.

In some embodiments, the information alerts the user to take a given medication according to a select schedule. In some embodiments, a communication tag of the communication tags provides one or more characteristics of the module of the plurality of modules. In some embodiments, the one or more characteristics indicate when the module is removed from the body and when the module is placed in the body. In some embodiments, the one or more proximity sensors detects the presence of the user when a distance between the body and the user is smaller than a predetermined distance. In some embodiments, the predetermined distance is at most about 1 meter. In some embodiments, the predetermined distance is at most about 0.5 meter.

In some embodiments, the information is provided to the user on a mobile electronic device of the user. In some embodiments, the information is provided on a user interface of the mobile electronic device of the user. In some embodiments, the body further comprises a camera configured to detect a presence of medication in a compartment of the one or more compartments. In some embodiments, the method further comprises providing the user with an alert indicating the presence of medication is detected in the compartment after a time period for the user to take the medication. In some embodiments, a compartment of the one or more compartments comprises an indicator, and wherein the indicator indicates that medication is to be taken from the compartment. In some embodiments, the method further comprises using a sorting unit to sort the medication into the one or more compartments.

In another aspect, the present disclosure provides a method for providing a medication to a user, comprising: providing a smart health management device comprising (i) a body, (ii) a locking unit, and (iii) one or more user identification units, wherein the body comprises a base and a plurality of modules, wherein the locking unit locks the plurality of modules to the base, and wherein a module of the plurality of modules holds the medication for use by the user; using the one or more user identification units to identify the user; and upon identifying the user, directing the locking unit to unlock the module from the base, thereby permitting the user to remove the module from the base when the module is unlocked from the base.

In some embodiments, the plurality of modules comprises one or more compartments, and wherein the one or more compartments hold the medication for use by the user. In some embodiments, the body further comprises a camera that detects a presence of medication in the module. In some embodiments, the method further comprises providing the user with an alert indicating the presence of medication is detected in the module after a time period for the user to take the medication. In some embodiments, the module comprises one or more indicators, and wherein the one or more indicators indicate that medication is to be taken from the module. In some embodiments, the module is locked to the base such that a user cannot access the medication in the module. In some embodiments, the one or more identification units identify the user by facial recognition, finger printing, voice recognition, or personal identification number. In some embodiments, the locking unit unlocks a single module at a time. In some embodiments, the method further comprises displaying information to a user on a display on the body. In some embodiments, the information comprises dosing information, dosing schedule, and/or patient education. In some embodiments, the method further comprises providing audio indication of dosing information and/or dosing schedule via a speaker on the body.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “figure” and “FIG.” herein), of which:

FIG. 1A shows an example of an industrial configuration of a body of a smart health management device; FIG. 1B shows an example of an industrial configuration of the smart health management device;

FIG. 2A shows a first view of another example of an industrial configuration of a smart health management device; FIG. 2B shows a second view of another example of an industrial configuration of the smart health management device;

FIG. 3 is an example of a block diagram illustrating how a smart health management device functions;

FIG. 4 shows a plurality of graphical user interfaces (GUIs) as part of an application that is executed on a mobile electronic device of a user;

FIGS. 5A-5B show schematics of a configuration of the smart health management device; FIG. 5A shows a schematic of a configuration of a base and corresponding modules; FIG. 5B shows a schematic of a configuration of a module and compartments;

FIG. 6 shows an example schematic of communication pathways between components of a smart health management device and one or more external components;

FIGS. 7A-7E show an example smart health management device; FIG. 7A shows an example external housing a health management device; FIG. 7B shows an example health management device with cover removed; FIG. 7C shows an example of the internal structure of an example health management device; FIG. 7D shows an example health management device with modules inserted; FIG. 7E shows an example of a module of a health management device; and

FIG. 8 shows a computer system that is programmed or otherwise configured to implement devices and methods of the present disclosure.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

The term “medication adherence,” as used herein, generally refers to the extent to which subjects, such as patients, follow provider recommendations with respect to the timing, dosage, and/or frequency of medication. Medication adherence may be the extent to which a subject, such as a patient, acts in accordance with the prescribed interval and dose of a dosing regimen.

The term “short-range communication,” as used herein, generally refers to a communication protocol that enables at least two devices, such as portable devices (e.g., a smart phone and a smart health management device), to establish communication with and communicate with one another. Short-range communication may include near field communication, Bluetooth communication, WiFi, or any other short-range communication protocol. Short-range communication may enable communication between electronic devices within about 10 meters, 8 meter, 6 meters, 5 meters, 4 meters, 3 meters, 2 meters, 1 meter, or less of one another.

The term “near field communication” (NFC), as used herein, generally refers to a communication protocol that enables at least two devices, such as portable devices (e.g., a smartphone), to establish communication with and communicate with one another. NFC may enable communication between electronic devices in proximity (e.g., at most 5 meters, 4 meters, 3 meters, 2 meters, 1 meter, or less) to one another. The NFC technology allows a device, such as a reader, interrogator, or active device, to create a radio frequency current that communicates with another NFC compatible device or a small NFC tag or chip having the information for transmission to the reader. Passive devices, such as the NFC tag in smart posters, for example, may store information and communicate with the reader but may not actively read other devices. Peer-to-peer communication through two active devices is also possible with NFC.

The term “proximity sensor,” as used herein, generally refers to a sensor capable of detecting the presence of nearby objects, in some cases without any physical contact. A proximity sensor may emit an electromagnetic field or a beam of electromagnetic radiation (infrared, for instance), and looks for changes in the field or return signal. The object being sensed may be referred to as a target of the proximity sensor. Different proximity sensor targets may require different sensors.

The term “user identification unit,” as used herein, generally refers to a system or method for identifying a user. A user identification unit may identify a user by facial recognition, voice recognition, fingerprint, and/or personal identification code. For example, the health management device may have a camera and may recognize and identify a user through facial recognition or fingerprint. Alternatively, or in addition to, the health management device may have a microphone and may recognize and identify a user through voice recognition.

The term “magnetic sensor,” as used herein, generally refers to a sensor capable of detecting changes and disturbances in a magnetic field, such as a change in flux, strength and/or direction of the magnetic field. The terms “include” and “comprise” can be used synonymously. The terms “subject” and “user” can be used synonymously.

The term “relay,” as used herein, generally refers to an electrically operated switch. A relay may use an electromagnet to mechanically operate the switch. The relay may be used in instances in which it may be necessary to control a circuit by a separate low-power signal, or situations in which several circuits are controlled by one signal. The term “smart,” as used herein, generally refers to a device or system feature that may be employed with reduced or minimal involvement by a user, such as through the use of computer intelligence (e.g., artificial intelligence).

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

### Smart Health Management Devices and Methods of Use

In an aspect, the present disclosure provides a smart health management system comprising a smart health management device. The smart health management device may comprise a body having a plurality of modules or compartments. A module of the plurality of modules may comprise one or more compartments. The compartments may be configured to hold or may hold medication for use by a user. The smart health management device may further comprise one or more proximity sensors, a short-range communication interface, and one or more computer processors. The one or more proximity sensors may be configured to detect or may detect a presence of a user in proximity to the smart health management device. The short-range communication interface may be configured to detect or may detect communication tags (e.g., near field communication (NFC) tags) attached to the plurality of modules. The one or more computer processors may be configured or otherwise programmed to (i) use the one or more proximity sensors to detect the presence of the user in proximity to the body, (ii) use the short-range communication interface (e.g., NFC interface) to detect the communication tags (e.g., NFC tags), thereby detecting the plurality of modules, and (iii) upon detecting the plurality of modules, provide the user with information corresponding to the medication in the one or more compartments when the user is in proximity to the body of the smart health management device.

In another aspect, the present disclosure provides a smart health management device, comprising a body, a locking unit, one or more user identification units, and one or more computer processors. The body may comprise a base and a plurality of modules. A module of the plurality of modules may be configured to hold medication for use by a user. The locking unit may be configured to lock the plurality of modules to the base. The locking unit may be configured to lock one or a subset, but not all, of plurality of modules to the base. The one or more user identification units may be configured to or may identify the user. The one or more computer processors may be configured or otherwise programmed to (i) use the one or more user identification units to identify the user to permit the locking unit to unlock a module from the base and (ii) permit the user to remove the module from the base to thereby access the medication.

In another aspect, the present disclosure provides a method for providing a medication to a user. The method may comprise providing a smart health management device comprising (i) a body having a plurality of modules, (ii) one or more proximity sensors that detect a presence of a user in proximity to the body, and (iii) a short-range communication interface that detects communication tags attached to the plurality of modules. A module of the plurality of modules may comprise one or more compartments that hold the medication for use by the user. The one or more proximately sensor may be used to detect the presence of the user in proximity to the body. The short-range communication interface may be used to detect the communication tags thereby detecting the plurality of modules. The smart health management device may provide the user with information corresponding to the medication in the one or more compartments when the user is in proximity to the body.

In another aspect, the present disclosure may provide a method for providing medication to a user. The method may comprise providing a smart health management device comprising (i) a body, (ii) a locking unit, and (iii) one or more user identification units. The body may comprise a base and a plurality of modules. The locking unity may lock the plurality of modules to the base. A module of the plurality of modules may hold the medication for use by the user. The one or more user identification units may be used to identify a user. Identification of the user may permit the locking unit to unlock the module from the base. The locking unit may unlock one or a subset, but not all, of the plurality of modules from the base. Unlocking the module from the base may permit the user to remove the module from the base to thereby access the medication.

The body may be in a shape of a circle, a diamond, a square, a triangle, a rectangle, an oval, a hexagon, a pentagon, or a partial shape or a combination of shapes thereof. Suitable materials of the body may include metal, plastic, steel, wood, glass, a composite, or other materials. The color of the body may be any color or any combination of colors, such as, for example, white, black, red, blue, yellow, or green. A lower end of the body may be flat or substantially flat, which may allow the user of the smart health management device to steadily place the body on a flat surface. The body may have a contemporary and slim configuration with soft curves.

The smart health management device may be used to store various articles, such as medication, vitamins, food, or other objects. The smart health management device may be used to store medication and other articles, such as, for example, medication and vitamins. The smart health management device may organize the medications and vitamins by when a user is scheduled to take the medications or vitamins. For example, the smart health management system may organize the medications and/or vitamins by day and further by time of day. In an example, a module may include the medications for a single day and a compartment may include the medications for a single time period during the day.

In an example, the smart health management device may comprise a plurality of compartments. The plurality of compartments may be protruded areas on a top end of the body. The plurality of compartments may be recessed areas on the top end of the body. The plurality of compartments may be flat or substantially flat areas on the top end of the body. A given compartment of the plurality of the compartments may further comprise a recessed area in the middle of the given compartment. The recessed area in the middle of the given compartment may be configured to hold a container in place. The plurality of compartments may be evenly distributed along a perimeter line of the body. The number of compartments may be at least 2, 3, 4, 5, 6, 7, 8, 9, 10, 11, 12, 13, 14, 15, 16, 17, or greater. The shape of one of the compartments may be a circle, a diamond, a square, a triangle, a rectangle, an oval, a hexagon, or a pentagon, or a partial shape or combination of shapes thereof.

Alternatively, or in addition to, the smart health management device may comprises a plurality of modules. A health management device may comprise greater than or equal to 2, 3, 4, 5, 6, 7, 8, 9, 10, 12, 15, 20, 25, 30, or more modules. In an example, a health management system may have seven modules and each module may represent a day of the week. A module may comprise one or more compartments. The module may comprise greater than or equal to 1, 2, 3, 4, 5, 6, 7, 8, 9, 10, 12, 15, 20, 25, 30, or more compartments. In an example, each module has four compartments and each compartment may represent a time of the day when a medication is to be taken. The health management device may comprise a base and the modules may be removable from the base. Alternatively, or in addition to, the modules may not be removable from the base. The module may be divided into the compartments. Each compartment may be accessed individually, or alternatively, multiple compartments may be accessed at one time. The plurality of modules may protrude from a top end of the body. The plurality of modules may be recessed areas on the top end of the body. The plurality of modules may be evenly distributed along a perimeter line of the body. The shape of one of the modules may be a circle, a diamond, a square, a triangle, a rectangle, an oval, a hexagon, or a pentagon, or a partial shape or combination of shapes thereof. In an example, a module has a shape of a triangular prism.

A cross-section of a shape may be at most 10 centimeters (cm), 5 cm, 4 cm, 3 cm, 2 cm, 1.5 cm, 1.2 cm, 1 cm, 0.9 cm, 0.8 cm, 0.7 cm, 0.6 cm, 0.5 cm, 0.1 cm, or less. For example, if the shape of one of the compartments is a circle, the radius of the circle may be at most 10 cm, 5 cm, 4 cm, 3 cm, 2 cm, 1.5 cm, 1.2 cm, 1 cm, 0.9 cm, 0.8 cm, 0.7 cm, 0.6 cm, 0.5 cm, 0.1 cm, or less. As another example, if the shape of one of the compartments is a square, the length of the side may be at most 10 cm, 5 cm, 4 cm, 3 cm, 2 cm, 1.5 cm, 1.2 cm, 1 cm, 0.9 cm, 0.8 cm, 0.7 cm, 0.6 cm, 0.5 cm, 0.1 cm, or less.

The plurality of compartments may be configured to hold containers containing medication for use by a user. Alternatively, or in addition to, the plurality of compartments may hold the medication for use by a user. The medication may be an over-the-counter medicine, prescribed medicine, vitamin, mineral supplement, or dietary supplement. The form of the medication may be a pill, capsule, caplet, powder, liquid, or aerosol. The containers may be organized on the compartments by time period, such as, for example, day of a week, day of a month, or month of a year.

The containers may be pill bottles that subjects (e.g., patients) receive from hospitals, pharmacies, clinics after their doctor's appointments. At least a subset of the containers may be pill bottles. The containers may be pill organizers that subjects bought online or from stores. The containers may be provided by the manufacture of the smart health management device. If the containers are provided by the manufacture of the smart health management device, the containers may be irremovably attached to the smart health management device and may not be removed from the smart health management device. In this situation, the subject may only open and close the containers. The container may be irremovably attached by, for example, glue, contact cement, epoxy, or any other type of adhesive. If the containers are provided by the manufacture of the smart health management device, the containers may be removably attached to the smart health management device, which may permit the containers to be removed from the smart health management device. The container may be removably attached to the smart health management device by, for example, a magnet or a clamping unit that permits a container to be attached to or removed from the smart health management device by snapping, prying, twisting, popping, levering, screwing, turning, or rotating. In such situation, the subject may not only open and close the containers, but also remove the containers. The containers may be pill bottles provided by the manufacture of the smart health management device. The containers may be reduced-size pill bottles that are suitable for travelling. The shape of the bottom end of one of the containers may be the same as the shape of at least one of the compartments. The shape of the bottom end of one of the containers may be different from all of the compartments. The size of the bottom end of one of the containers may be the same as or smaller than the size of at least one of the compartments. The size of the bottom end of one of the containers may be bigger than the size of all of the compartments. The shape of one of the containers may be, for example, a cylinder, a cone, a cube, or a sphere. Alternatively, or in addition to, the smart health management system may not hold a plurality of containers and the compartments may hold the medication.

The smart health management device may comprise a plurality of light emitting diode (LED) notification structures. The shape of one of the plurality of LED notification structures may be a ring, an arc of a circle, a diamond, a square, a triangle, a rectangle, an oval, a hexagon, or a pentagon. One or more LED notification structures of the plurality of LED notification structures may be placed on the top of or in proximity to a module of the plurality of modules or a given compartment of the plurality of compartments. One or more LED notification structures placed on the top of or in proximity to a given module or compartment may be activated when a given container is placed on the given compartment. Alternatively, or in addition to, one or more LED notification structures placed on top of or in proximity to a given module or compartment may be activated with a medication is placed in the module of compartment. When activated, the one or more LED notification structures placed on the top of or in proximity to a given module or compartment may demonstrate one or more colors. The one or more colors may include red, blue, yellow, pink, or any color. The different colors of the one or more LED notification structures may indicate different information of the given container. For example, a red color may demonstrate the given container is placed on a wrong compartment, and a green color may demonstrate that the given container is placed on the right given compartment.

The smart health management device may further comprise one or more proximity sensors. The one or more proximity sensors may be placed on the top end of the body. The one or more proximity sensors may be placed at the bottom end of the body. The one or more proximity sensors may be positioned at the center of the body. The one or more proximity sensors may be imbedded inside the body so that the user cannot observe them. The one or more proximity sensors may convert information on the movement or presence of the user into an electrical signal. The one or more proximity sensors may use the eddy currents to sense the user by electromagnetic induction. The one or more proximity sensors may detect changes in electrical capacity when the user is approaching. Alternatively, or in addition to, the one or more proximity sensors may be motion sensors that detect motion of a user when in proximity to the smart health management device. The one or more proximity sensors may use magnets and/or reed switches.

The one or more proximity sensors may be configured to detect a presence of the user in proximity to the body. The one or more proximity sensors may be configured to detect the presence of the user when a distance between the body and the user is smaller than a predetermined distance. The predetermined distance may be at most 5 meters (m), 2 m, 1 m, 0.9 m, 0.8 m, 0.7 m, 0.6 m, 0.5 m, 0.4 m, 0.3 m, 0.2 m, 0.1 m, 0.05 m, 0.01 m, or less. Once the one or more proximity sensors detect the user in proximity to the body, the user may receive information regarding to the user's medication. The information may alert the user to take a given medication according to a selected schedule. The information may further comprise the name of the medication, the function of the medication, the remaining dose of the medication, the time for a scheduled dose, side effects and other information related to the user or the medication.

The smart health management device may further comprise a short-range communication interface. In an example, the short-range communication interface is a near field communication (NFC) interface. The standards of the NFC interface may include, but not limited to, ISO/IEC 18092. The NFC interface may be used to engage in near field communication with other NFC enabled devices. Alternatively, or in addition to, the short-range communication interface may include Bluetooth, WiFi, or inter-integrated circuit (I²C) communication interfaces. The short-range communication interface may be used to engage short-range communication with other short-range communication enabled devices. The short-range communication enabled devices may include mobile phones, PCs, tablets, printers, consumer electronics, smartwatches, and appliances.

The short-range communication interface may be configured to detect short-range communication tags attached to the modules or containers. Although “tags” are used to describe short-range communication targets, any types of short-range communication targets may be used, such as short-range communication coasters, short-range communication stickers, short-range communication fobs, or short-range communication cards. One or more of the short-range communication tags may be attached to one or more of the modules or containers. A given short-range communication tag of the short-range communication tags may be attached to a part of a given module of the modules or container of the containers. The part of the given module of container may be the bottom end, the top, the outside, the inside, or anywhere of the given module or container. The mechanism of the given short-range communication tag's attachment to the given module or container may be Velcro and/or any other mechanisms. Each of the short-range communication tags may comprise one or more short-range communication passive antennas, for example, an NFC passive antenna. The NFC passive antennas may be powered by the NFC interface that generates a radio frequency.

The short-range communication interface may establish a short-range communication connection with the short-range communication tags. The short-range communication connection may be established when the short-range communication tags are placed within a predetermined distance from the short-range communication interface. The predetermined distance may be at most 5 m, 2 m, 1 m, 0.1 m, 0.05 m, 0.01 m, 0.5 cm, 0.1 cm, or less. Prior to establishing the short-range communication connection, the short-range communication interface may be inactive. Once the short-range communication connection is established, the short-range communication interface may communicate information with the short-range communication tags through the short-range communication connection. In an example, the short-range communication interface is a NFC interface and the short-range communication tags are NFC tags.

The information may comprise one or more characteristics of a given container of the containers. A given short-range communication tag of the short-range communication tags may provide the one or more characteristics of the given module of the modules or container of the containers. The one or more characteristics may indicate when the given module or container is removed from the health management device, and when the given module or container is placed in the health management device. The one or more characteristics may be amount, quantity, mass, weight, and/or volume of the given container or medication inside the given container or module. The information may alert the user to take a given medication according to a selected schedule. The information may further comprise the name of the medication, the function of the medication, the remaining dose of the medication, the time for a scheduled dose, side effects and other information related to the user or the medication.

The smart health management device may comprise one or more magnetic sensors. The one or more magnetic sensors may be placed on the top end of the body. The one or more magnetic sensors may be placed at the bottom end of the body (e.g., on or around the base). The one or more magnetic sensors may be imbedded inside the body so that the user cannot observe them. The magnetic sensors may be used to hold a module in the base or a given container on the top of a given compartment. The magnetic sensors may be configured to detect a given container when the given container is placed in the given compartment.

Each of the short-range communication tags may further comprise one or more magnets. A given short-range communication tag of the short-range communication tags with one or more magnets may be attached to the bottom end of a given container of the containers. When placing the given container in proximity to the body of the smart health management device, the given short-range communication tag with one or more magnets may interact with one or more magnetic sensors of the body through a magnetic field. The magnetic field may assist holding the given container on the top of the given compartment of the plurality of the compartments.

As an alternative to an NFC tag, any other approach for identifying the containers may be used. The container may be identified by various types of tags. For example, a tag may be a radio-frequency identification (RFID) tag or a chip that permits communication using WiFi or Bluetooth (e.g., Bluetooth Low Energy). The container may also comprise a barcode that can be scanned by an electronic device, which may permit the container to be identified.

The smart health management device may comprise one or more locking units. The locking units may be configured to or may lock a module to the base, a compartment to prevent a user from accessing the compartment, or a container to the compartment. The locking units may be configured such that only a single module may be unlocked and removed at a time. Additionally, the locking units may lock the compartments such that a single compartment may be opened at a time. Alternatively, or in addition to, multiple compartments may be unlocked and opened at a time. The locking unit may be a normally closed lock such that, in the event of a device failure, the modules and compartments are locked. Alternatively, or in addition to, the locking unit may be a normally open lock such that, in the event of a device failure, the modules and compartments are not locked. The locking unit may be a mechanical lock, electronic lock, magnetic lock, or any combination thereof.

The smart health management device may comprise one or more user identification units. The user identification units may be disposed in the body of the smart health management device. Alternatively, or in addition to, the user identification units may be disposed on the modules. The user identification unit may identify a user. User identification may permit a user to access medications stored within the smart health management device. The user identification unit may identify a user by facial recognition, voice recognition, fingerprinting, or via a unique identification code. The smart health management device may recognize at least 1, 2, 3, 4, 5, 6, 8, 10, or more different users. A user may be the person taking the medication or a caretaker of the person taking the medication.

The smart health management device may comprise a sorting unit configured to sort the medication into the modules or compartments. Alternatively, or in addition to, the sorting unit may be separable from the smart health management device and may be coupled to the smart health management device to load medications into the device. The sorting unit may comprise one or more compartments such that a single medication is added to each compartment. The sorting unit may deliver medication from each of the compartments of the sorting unit to the compartments of the smart health management device. Alternatively, or in addition to, the medication may be added to a single compartment within the sorting unit and sorted based on mass, shape, color, barcode, numerical identifier, or any other identifying characteristic.

The smart health management device may further comprise a camera (e.g., CCD camera). The camera may be used for facial recognition or fingerprint recognition. Alternatively, or in addition to, the camera may be used to monitor contents of the modules or compartments. The modules or compartments may be at least partially transparent, and the camera may monitor the contents of the module and/or compartment. For example, if a user takes only a partial dose of medication, the camera may detect that a portion of the medication remains within the module or compartment.

The smart health management device may further comprise one or more computer processors (e.g., controllers) configured to use the one or more proximity sensors to detect the presence of the user in proximity to the body, use the one or more user identification units to identify a user, use the camera to monitor medication within a module and/or compartment, or lock and unlock the modules from the base. The range of sensing of the one or more proximity sensors may be at most 5 m, 2 m, 1 m, 0.9 m, 0.8 m, 0.7 m, 0.6 m, 0.5 m, 0.4 m, 0.3 m, 0.2 m, 0.1 m, 0.05 m, 0.01 m, or less. The computer processor may be positioned at the center of the body. The computer processor may be operatively coupled with one or more smart speakers. The one or more smart speakers may comprise, but not limited to, Alexa, Google Home, Google Assistant, Clova, Microsoft Cortana, AliGenie, Ambient, Apple HomeKit, Apple Siri, and Apple Pod.

The controller may be configured to use the short-range communication interface to detect the short-range communication tags. One or more of the short-range communication tags may be attached to one or more of the containers. Therefore, the controller may be configured to detect the containers. Upon detecting the containers, the controller may be configured to provide the user with information corresponding to the medication in the containers when the user is in proximity to the body. The controller may provide the user with the information on a mobile electronic device of the user. The mobile electronic device may include a mobile phone, PC, tablet, printer, consumer electronics, smartwatch, and appliance. The controller may provide the user with information on the mobile electronic device via cable, USB connection, Bluetooth, Bluetooth Low Energy, or Wi-Fi. The information on the mobile electronic device may be transferred to the controller via cable, USB connection, Bluetooth, Bluetooth Low Energy, or Wi-Fi. The information may be stored on a flash drive. The information on the flash drive may be transferred to the controller. The information may be provided on a user interface of the mobile electronic device. The information may be provided to the user of the smart health management device. The information may also be provided to other individuals that the user may select, including, but not limited to, user's friends, family members, and physicians.

The information may alert the user to take a given medication according to a selected or predetermined schedule. The information may further comprise the name of the medication, the function of the medication, the remaining dose of the medication, the time for a scheduled dose, side effects and other information related to the user or the medication. The information may further comprise one or more characteristics of a container. The one or more characteristics may indicate when the container is removed from a compartment and when the container is placed in the compartment. The one or more characteristics may be amount, quantity, mass, weight, and/or volume of the given container or medication inside the container.

The smart health management device may comprise a voice notification system. The voice notification system may allow the user to interact with the smart health management device through voice. The voice notification system may comprise a microphone, or a speaker. The speaker may be a smart speaker. The smart speaker may be, but not limited to, Alexa, Google Home, Google Assistant, Clova, Microsoft Cortana, AliGenie, Ambient, Apple HomeKit, Apple Siri, or Apple Pod. The smart health management device may be integrated with different services. The services may include, but not limited to, Alexa Voice Services and Google Voice. The smart health management device may be activated with a wake word. The wake word may be a customized wake word or a word pre-determined by the services. The services may allow the user to control the smart health device through voice. The services may allow the user to provide inputs with voice on different tasks, for example, but not limited to, rescheduling the medication reminder, refilling prescriptions and vitamins, diet, mood, symptoms, and biomarkers such as glucose levels or blood pressure.

The voice notification system may be operatively coupled with the proximity sensors and/or user identification unit to provide the user with information regarding the medication or health of the user. For example, when an individual is within a predetermined distance from the proximity sensors of the smart health management device, the proximity sensors may first verifying that the individual is the user of the smart health management device. If the proximity sensors verify that the individual is the user of the smart health management device, the voice notification system operatively coupled with the proximity sensors may be activated to provide voice output to the user and allow the user to interact with the smart health management device.

The interaction between the user and the smart health management device may include rescheduling a reminder or alert to a later time, switching off the reminder or alert, or recording medications with the pharmacies. The smart health management device may allow the user to schedule the time at which he/she may take medications. The voice notification system may notify or alert the user through voice to take a given medication according the schedule.

The voice notification system may respond to the user's inquiries about the medication in the containers. The user's inquiries may be any information regarding the medication. The user's inquiries may be related to recent news about the medication. The recent news about the medication may be obtained from journal articles, websites, television, or any approach that may inform the general public about the medication. The recent news about the medication may comprise additional symptoms triggered by the medication, warnings of recent deaths, latest research on the medication or relevant conditions, and any updates about the medication. The user's inquiries may include an amount, quantity, mass, weight, and/or volume of the given container or medication inside the given container. The user's inquires may further include the name of the medication, the function of the medication, the remaining dose of the medication, the time for a scheduled dose, side effects and other information related to the user or the medication. The voice notification system may remind the user of his/her exercise schedule and provide active lifestyle suitable for the user. The voice notification system may provide the user with information about healthy meal ideas. The voice notification system may provide inputs on the user's blood pressure and glucose concentration. The voice notification system may provide the user with information of the user's water intake. The voice notification system may comprise, for example, but not limited to, Alexa Voice Services or Google Voice.

The smart health management device may further comprise a relay to switch between the medications. The smart health management device may comprise a plurality of antennas. The plurality of antennas may be placed in a plurality of compartments and/or a plurality of containers of the smart health management device. Each of the compartments and/or each of the containers may comprise one or more antennas. The relay may cycle through the plurality of antennas to check if one or more containers are taken by the subject. Whether the one or more containers are taken by the subject may represent whether one or more medications are taken by the subject. The smart health management device may be powered by DC power, AC power, or batteries. The smart health management device may be powered through a USB connection by DC power, AC power, or batteries.

A smart health management system may comprise the smart health management device to drive adherence, awareness and accountability of the subject towards improving his/her health outcome. The smart health management system may be configured to perform a plurality of actions to influence the subject to adjust different aspects of his/her lifestyle and improve his/her health. The plurality of actions may include, but not limit to, reminding and monitoring the user's schedule to take medications, providing relevant information regarding the medications, allowing the user to set exercise goals, creating exercise schedule for the user, reminding the user of his/her exercise schedule, accessing whether the user meets his/her exercise goals, providing active lifestyle suitable for the user, monitoring the user's heart rate, blood pressure and glucose concentration, detecting the user's mood and/or sleep pattern, checking user's water intake, allowing individuals selected by the user (e.g. physicians, friends, or family members) to communicate with the user about his/her health improvement, and providing the user with nutrient information and healthy meal ideas.

The smart health management system may allow the smart health management device to connect to electronic devices. The electronic devices may be wearable devices, including but not limited to, Fitbit, Apple watch, Samsung health, Misfit, Xiaomi Mi band, and Microsoft band. The smart health management device may connect to the electronic devices to import data from the electronic devices. The data may comprise, but not limited to, exercise regime, goals, number of steps, heart rate, blood pressure, weight, and glucose levels.

The smart health management system may be configured to perform the plurality of actions through one or more algorithms. The one or more algorithms may be implemented by one or more software programs. The one or more algorithms may influence the user's behavior and persuade the user to take actions regarding his/her health. The one or more algorithms may, but not limited to, facilitate the communications between the voice notification system with the user, permitting other individuals selected by the user (e.g. physicians, friends, or family members) to communication with the user, providing information about the effect of the medications on various aspects related to the user's health, and allow the user to control the smart health management device through the electronic devices. The one or more algorithms may also identify correlations between the user's failure to take medications and the user's other actions and characteristics. The user's other actions and characteristics may comprise, but not limited to, the user's exercise regime, the number of steps finished by the user during the exercise, the user's heart rate, the user's weight, the user's blood pressure, the user's glucose levels, and the user's sleep pattern. The one or more algorithm may also identify the correlations between the user's medication adherence and the frequency of the user's exercise. The one or more algorithm may also provide insights into the user's medication adherence over a period of time. The period of time may be at least two days, one week, one month, one quarter, half a year, one year, or longer. The one or more algorithms may be machine learning algorithms.

Reference will now be made to the figures, wherein like numerals refer to like parts throughout. It will be appreciated that the figures and features therein are not necessarily drawn to scale.

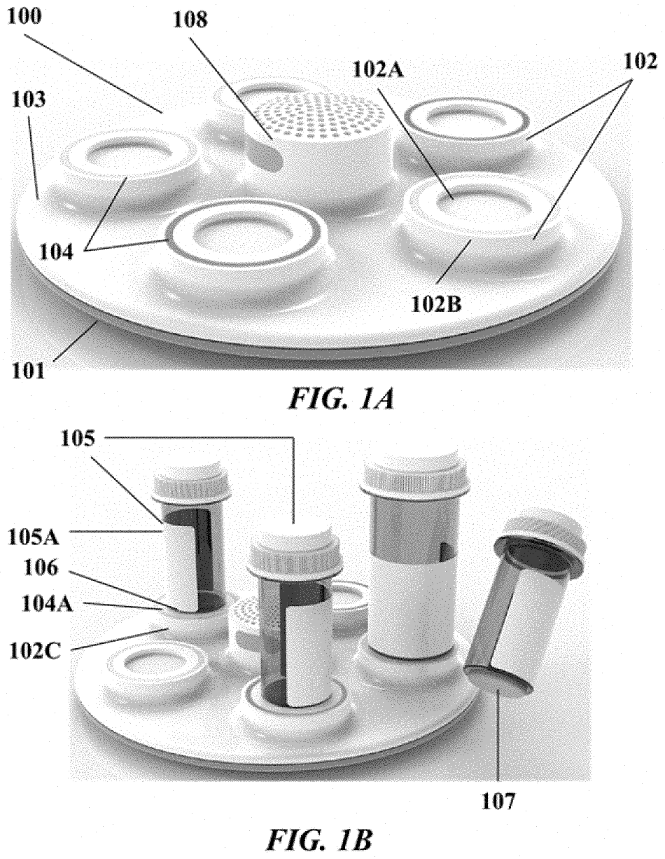

FIG. 1A shows an example of an industrial configuration of a body of a smart health management device. FIG. 1B shows an example of an industrial configuration of the smart health management device.

The smart health management device may include a body 100. In this illustrated example, the shape of the body 100 is a circle and the color of the body 100 is white. Suitable material of the body 100 may include metal, plastic, steel, wood, glass, and/or other materials. A lower end 101 of the body may be flat, which may allow a user of the smart health management device to steadily place the body 100 on a flat surface. The body 100 may have a contemporary and slim configuration with soft curves.

The body of the smart health management device 100 may comprise a plurality of compartments 102. In the illustrated example, the plurality of compartments 102 are protruded areas on the top end 103 of the body 100, the number of the compartments is 5, and the shape of the compartments 102 is circle. The radius of the compartments 102 may be at most 10 cm, 5 cm, 3 cm, 2 cm, 1.5 cm, 1.2 cm, 1 cm, 0.9 cm, 0.8 cm, 0.7 cm, 0.6 cm, 0.5 cm, 0.1 cm, or less. A given compartment 102B of the plurality of the compartments in the illustrated example further comprises a recessed area 102A in the middle of the given compartment. The recessed area 102A may be configured to hold a container in place. The plurality of compartments 102 in the illustrated example are evenly distributed along the perimeter line of the body 100.

The plurality of compartments 102 may comprise one or more LED notification structures 104. The LED notification structures 104 in the illustrated example are placed on the top of the plurality of compartments. The shape of the LED notification structures 104 in the illustrated example is ring.

The plurality of compartments 102 may be configured to hold containers 105 containing medication for use by a user. The containers 105 in the illustrated example are pill bottles that a subject receives from hospitals, pharmacies, clinics after their doctor's appointments. The shape of the bottom end 106 of the container 105A in the illustrated example is the same as the shape of the compartment 102C. The shape of the containers 105 in the illustrated example is a cylinder. When the container 105A in the illustrated example is placed on the compartment 102C, the LED notification structure 104A is activated. When activated, the LED notification structure 104A demonstrates one or more colors. The one or more colors may be red, blue, yellow, pink, or any color.

The body 100 of the smart health management device may further comprise one or more proximity sensors, a controller, an NFC interface, and/or one or more magnetic sensors. In the illustrated example, the proximity sensors, the controller, the NFC interface, and/or the magnetic sensors are imbedded inside the body so that the user cannot observe them. The NFC interface may establish an NFC connection with the NFC tags. One of the NFC tags 107 in the illustrated example is attached to the bottom end of a container. The mechanism of the attachment to the container may be Velcro and/or any other mechanisms. The NFC tags may further comprise one or more magnets, which, in the illustrated example, are imbedded inside the NFC tags so that the user cannot observe them. The size of the NFC tag 107 may be the same as or smaller than the size of the recessed area 102A in the middle of the compartment 102B.

The smart health management device may comprise a voice notification system 108. In the illustrated example, the voice notification system 108 is placed at the center of the body 100. The voice notification system may allow the user to interact with the smart health management device through voice. The voice notification system may comprise a microphone or a speaker.

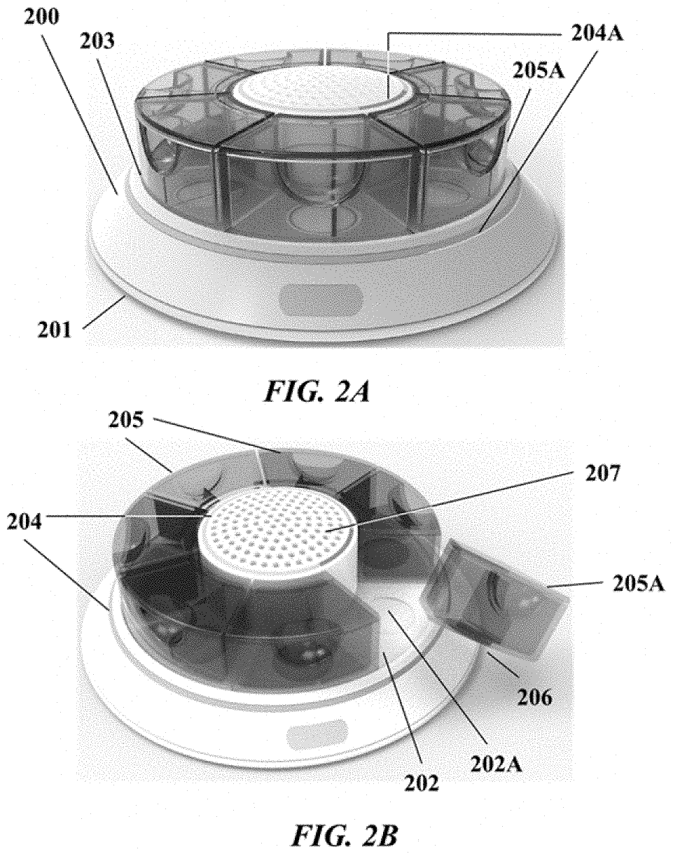

FIG. 2A shows a first view of another example of an industrial configuration of a smart health management device. FIG. 2B shows a second view of another example of an industrial configuration of the smart health management device. The smart health management device may include a body 200. The shape of the body 200 in the illustrated example is a circle. Suitable material of the body 200 may include metal, plastic, steel, wood, glass, and/or other materials. The color of the body 200 in the illustrated example is white. The lower end 201 of the body may be flat, which allows the user of the smart health management device to steadily place the body 200 on a flat surface. The body 200 may have a contemporary and slim configuration with soft curves.

The body of the smart health management device 200 may comprise a plurality of compartments. The plurality of compartments in the illustrated example is the flat areas on the top end 203 of the body 200. A given compartment 202 of the plurality of the compartment in the illustrated example further comprises a recessed area 202A in the middle of the compartment 202. The recessed area 202A may be configured to hold a container in place. The number of compartments in the illustrated example is 7. The shape of the compartments in the illustrated example is an arc of a circular ring. The plurality of compartments may be evenly distributed along a perimeter line of the body 200.

The smart health management device may comprise a plurality of LED notification structures. The LED notification structures 204 in the illustrated example are placed in proximity to the plurality of compartments. The LED notification structures 204 in the illustrated example are placed at the edges of both a voice notification system 207 and the compartments. The shape of a given LED notification structure 204A in the illustrated example is an arc of a ring.

The plurality of compartments may be configured to hold containers 205 containing medication for use by a user. The containers 205 in the illustrated example are pill bottles provided by the manufacture of the smart health management device. The shape of the bottom end of the containers 205 in the illustrated example is the same as the shape of one of the compartments. The size of the bottom end of the containers 205 in the illustrated example is the same as the size of the compartments. The LED notification structure 204A may be activated when a given container 205A is placed on the given compartment. When activated, the given LED notification structure 204A may demonstrate one or more colors. The one or more colors may be red, blue, yellow, pink, or any color.

The body 200 of the smart health management device may further comprise one or more proximity sensors, a controller, an NFC interface, and/or one or more magnetic sensors. In the illustrated example, the proximity sensors, the controller, the NFC interface, and/or the magnetic sensors are imbedded inside the body so that the user cannot observe them. The NFC interface may establish an NFC connection with the NFC tags. One of the NFC tags 206 in the illustrated example is attached to the bottom end of a container. The mechanism of the attachment to the container may include Velcro and/or any other mechanisms. The NFC tags may further comprise one or more magnets, which, in the illustrated example, are imbedded inside the NFC tags so that the user cannot observe them. The body and/or the containers of the smart health management device may be portable so that the smart health management device may be used when the user is travelling.

The smart health management device may comprise a voice notification system 207. In the illustrated example, the voice notification system 207 is placed at the center of the body 200. The voice notification system may allow the user to interact with the smart health management device through voice. The voice notification system may comprise a microphone or a speaker.

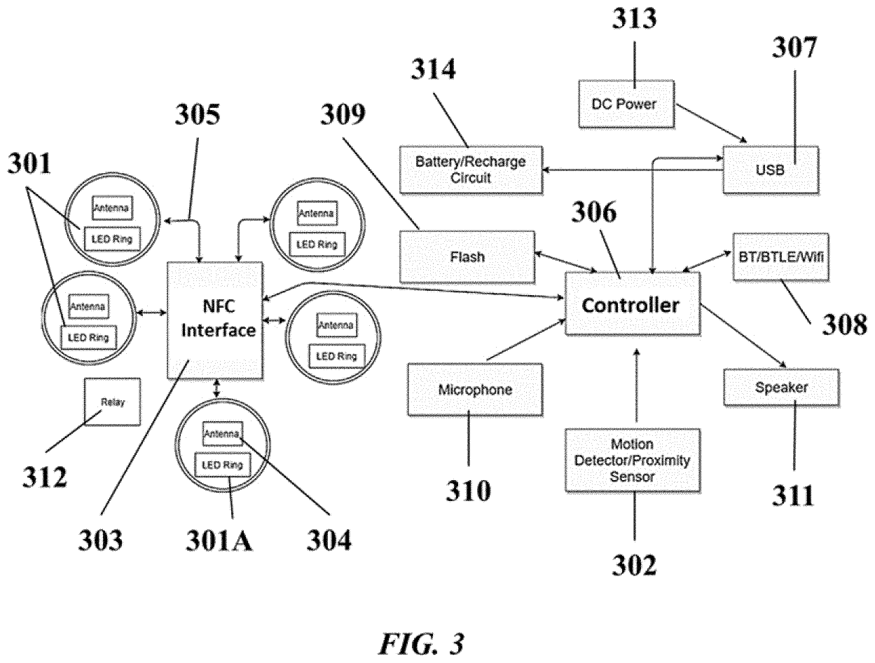

FIG. 3 is an example of a block diagram illustrating the manner in which a smart health management device of the present disclosure may function. The smart health management device may comprise one or more LED notification structures 301. A given LED notification structure 301A may be activated when a given container is placed on a given compartment of the smart health management device. When activated, the given LED notification structure 301A may demonstrate one or more colors. The one or more colors may be red, blue, yellow, pink, or any color. The different colors may indicate different characteristics of the given container.

The smart health management device may further comprise one or more proximity sensors 302. The one or more proximity sensors 302 may convert information on the movement or presence of the user into an electrical signal. The one or more proximity sensors 302 may use the eddy currents to sense the user by electromagnetic induction. The one or more proximity sensors 302 may detect changes in electrical capacity when the user is approaching. The one or more proximity sensors 302 may use magnets and reed switches.

The one or more proximity sensors 302 may be configured to detect the presence of the user in proximity to the body. The one or more proximity sensors 302 may be configured to detect the presence of the user when a distance between the body and the user is smaller than a predetermined distance. The predetermined distance may be at most 5 m, 2 m, 1 m, 0.9 m, 0.8 m, 0.7 m, 0.6 m, 0.5 m, 0.4 m, 0.3 m, 0.2 m, 0.1 m, 0.05 m, 0.01 m, or less. Once the one or more proximity sensors detect the user in proximity to the body, the user may receive information regarding to the user's medication.

The smart health management device may further comprise an NFC interface 303 (e.g., or other short-range communication interface). The standards of the NFC interface 303 may include, but not limited to, ISO/IEC 18092. The NFC interface 303 may be used to engage in near field communication with other NFC enabled devices. The NFC enabled devices may include mobile phones, PCs, tablets, printers, consumer electronics, and appliances. The NFC interface 303 may be configured to detect NFC tags attached to the containers. The NFC tags may comprise one or more NFC passive antennas 304. When the one or more NFC passive antennas 304 are placed in proximity to the NFC interface 303 and the LED notification structures 301, the NFC passive antennas 304 may be powered by the NFC interface 303 that generates a radio frequency.

The NFC interface 303 may establish an NFC connection 305 with the NFC tags through the NFC passive antennas 304. The NFC connection 305 may be established when the NFC interface 303 is placed within a predetermined distance from the NFC tags. The predetermined distance may be at most 5 m, 2 m, 1 m, 0.1 m, 0.05 m, 0.01 m, 0.5 cm, 0.1 cm, or less. Prior to establishing the NFC connection 305, the NFC interface 303 may be inactive. Once the NFC connection 305 is established, the NFC interface 303 may communicate information with NFC tags through the NFC connection 305.

The smart health management device may further comprise a controller 306 configured to use the one or more proximity sensors 302 to detect the presence of the user in proximity to the body. The controller 306 may be configured to use the NFC interface 303 to detect the NFC tags. One or more of the NFC tags may be attached to one or more of the containers. Therefore, the controller 306 may be configured to detect the containers. Upon detecting the containers, the controller 306 may be configured to provide the user with information corresponding to the medication in the containers when the user is in proximity to the body.

The controller 306 may provide the user with the information on a mobile electronic device of the user. The mobile electronic device may include a mobile phone, PC, tablet, printer, consumer electronics, and appliance. The controller may provide the user with information on a mobile electronic device via cable, USB connection 307, Bluetooth 308, Bluetooth Low Energy 308, or Wi-Fi 308. The information on a mobile electronic device may be transferred to the controller 306 via cable, USB connection 307, Bluetooth 308, Bluetooth Low Energy 308, or Wi-Fi 308. The information may be stored on a flash drive 309. The information on the flash drive 309 may be transferred to the controller 306. The information may be provided on a user interface of the mobile electronic device.

The information may alert the user to take a given medication according to a selected schedule. The information may comprise the name of the medication, the function of the medication, the remaining dose of the medication, the time for a scheduled dose, side effects and other information related to the user or the medication. The information may further comprise one or more characteristics of a given container. The one or more characteristics of a given container may be provided by a given NFC tag. The one or more characteristics may indicate when the given container is removed from a given compartment and when the given container is placed in the given compartment. The one or more characteristics may be amount, quantity, mass, weight, and/or volume of the given container or medication inside the given container.

The smart health management device may comprise a voice notification system. The voice notification system may allow the user to interact with the smart health management device through voice. The voice notification system may comprise a microphone 310, or a speaker 311.

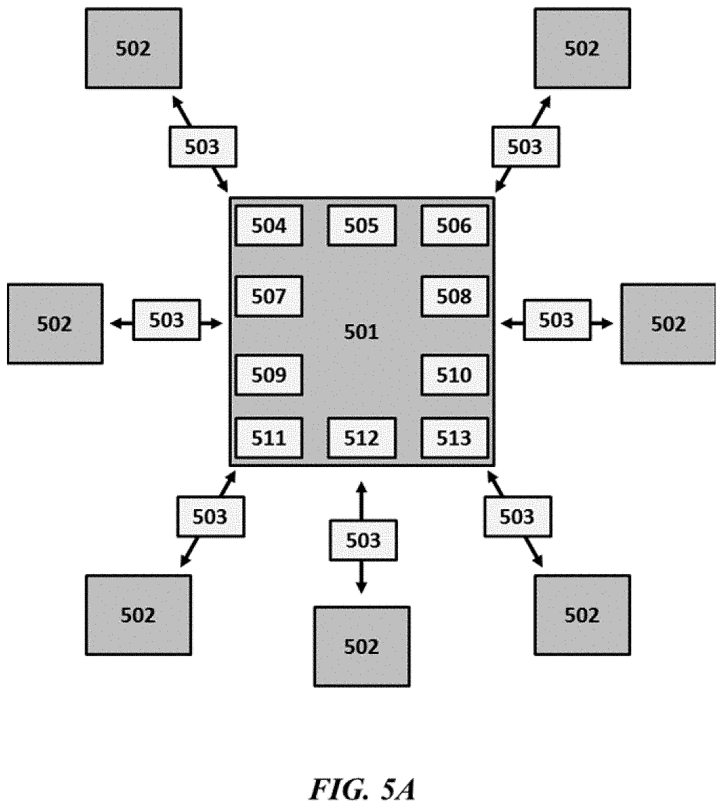

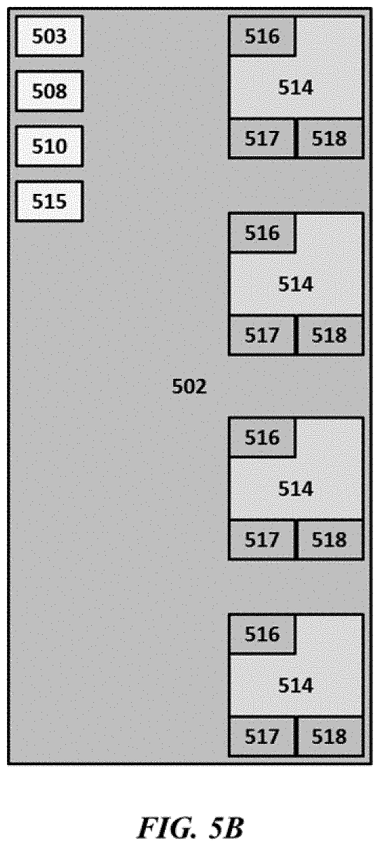

The smart health management device may further comprise a relay 312 to switch between the medications. The smart health management device may be powered by DC power 313, AC power, or batteries 314. The smart health management device may be powered through the USB connection 307 by DC power 313, AC power, or batteries 314. FIGS. 5A and 5B show a schematic of the smart health management device. FIG. 5A shows that the smart health management device may comprise a base 501 and one or more modules 502. The base 501 may be in communication with the modules 502 via a short-range communication interface 503. The base 501 may further comprise one or more computer processors (e.g., microprocessors) 504, locking units 505, user identification units 506, proximity sensors 507, camera(s) 508, microphone array 509, speaker 510, display 511, digital signal processor (DSP) 512, and/or automatic object sorting unit 513. FIG. 5B shows a schematic of an individual module 502. The module 502 may include multiple compartments 514, short-range communication tag 503, camera (e.g., CCD) 508, speaker 510, and an easy open mechanism 515. The camera 508 may sense if a full or partial dose of medication has been taken to ensure dosing compliance. Each compartment may include an LED 516, a magnet 517, and a hall effect sensor 518 for proximity sensing. The LED 516 may indicate which compartment 514 a user should take the medication from.

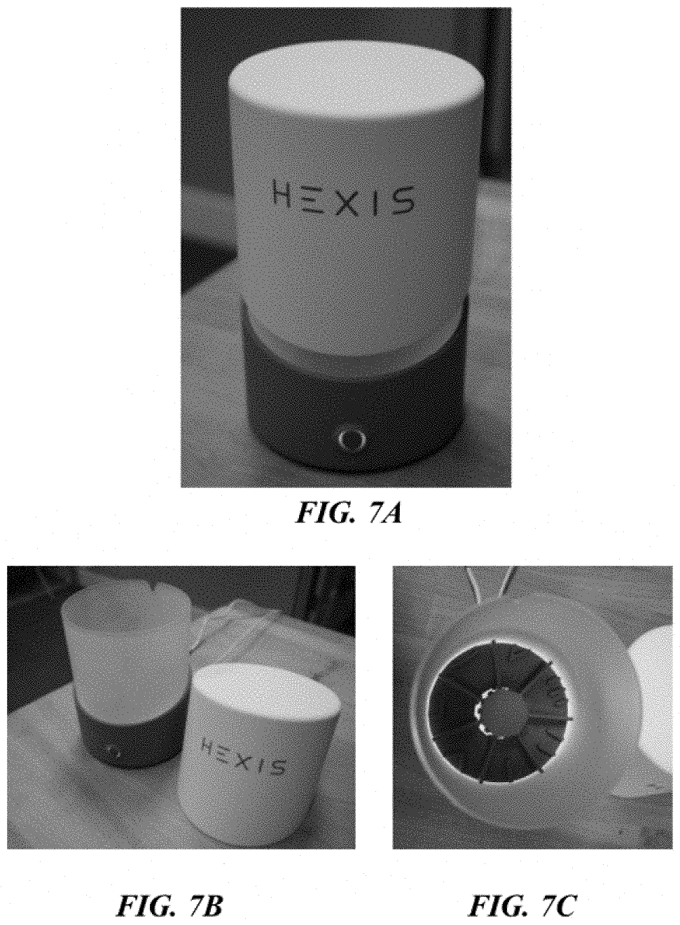

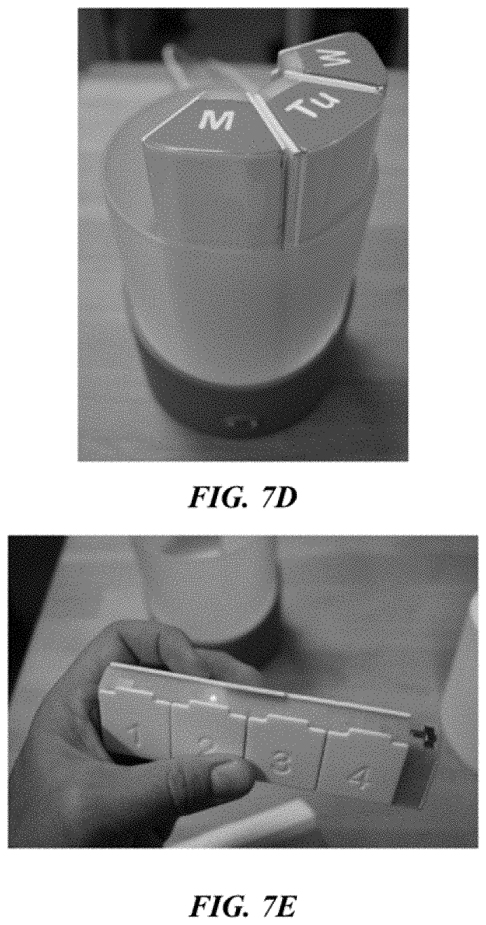

FIGS. 7A-7E show an example smart health management device. FIG. 7A shows an example external housing of a smart health management device. The external housing may have a removable lid or cover over the base. When the lid or cover is in place the modules of the device may not be visible. FIG. 7B shows the example smart health management device with the lid or cover removed from the base. The lid or cover may set in place or may be locked or latched such that only a user may remove the cover. FIG. 7C shows the internal structure of the base of the example smart health management device. The base may have one or more divisions or recesses in which the modules fit. The recesses may be configured to lock the modules to the base. Additionally, the base may be in communication with the modules such that the base senses or detects the presence or absence of the modules. FIG. 7D shows the example smart health management device with modules inserted. The modules may have a triangular prism shape and may be arranged radially around the center of the base. In the example, the base is configured to hold seven modules, one for each day of the week. FIG. 7E shows an example of a module of the example smart health management device. The module may have multiple compartments. In the example short, the module has four compartments. Each compartment may contain medications to be taken at a given time of the day. The module may have one or more LEDs. Each compartment may be associated with and LED. The LED may indicate which compartment to remove and take medication from. The compartments may be easy open such that those with low dexterity may open the containers to take the medication. The compartments may have a locking unit such that only a single compartment corresponding the given dosage schedule may be opened at a given time.

### User Interfaces

The present disclosure provides user interfaces (UIs) that may enable a user to interact with smart health management device provided herein. The UIs may be graphical user interfaces (GUIs).

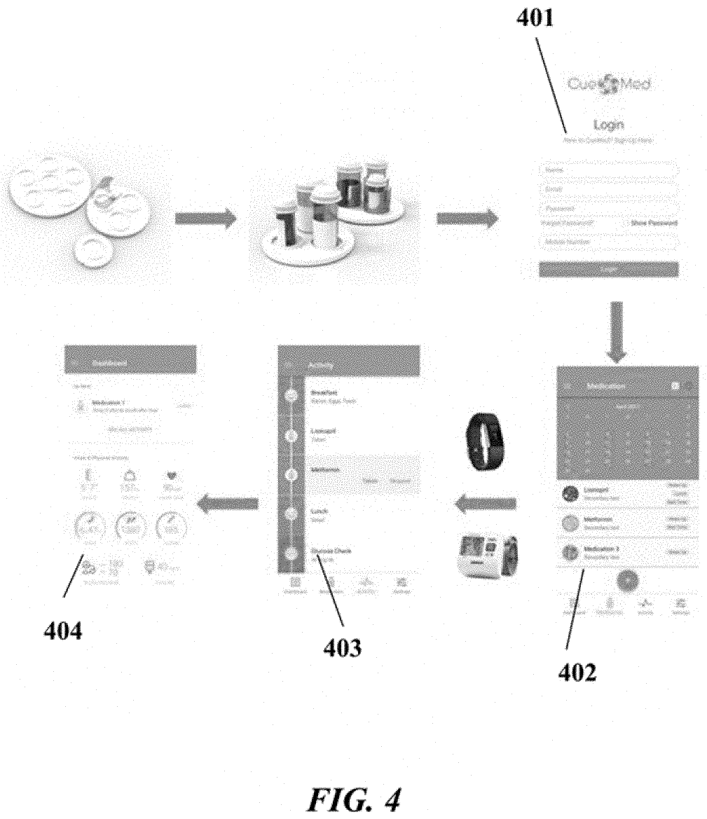

FIG. 4 shows an example of a plurality of GUIs as part of an application that may be executed on a mobile electronic device of a user. The application may be configured to control a smart health management device. The mobile electronic device may comprise an electronic display screen (“screen”) for presenting the GUIs.

The first GUI 401 may provide a login screen when the application is initiated. The user may choose either to enter his/her login information or to sign-up. If the user chooses to enter his/her login information, the GUI 401 may present a plurality of input fields. The input fields may allow the user to enter his/her name, email address, password, or mobile number. The GUI 401 may allow the user to choose to show his/her password when the password is entered. If the user forgets his/her password, he/she may click on an icon on the screen to retrieve his/her password.

The second GUI 402 may provide an example of a screen of information about user's medication. The second GUI 402 may comprise a plurality of graphical elements displayed on the screen. The plurality of graphical elements may comprise a calendar. The calendar may be a weekly calendar, a monthly calendar, or a quarterly calendar. The background color of the calendar may be gray, white, blue, red, or any color. The color of a date on the calendar may be different from the background color of the calendar. The date on the calendar may be marked to show the user that he/she needs to complete one or more activities on the marked date. The types of marks may include bold, underscore, highlight, or star. The calendar may be positioned at the top of the screen, the middle of the screen, the bottom of the screen, or a location that when the user is holding the device, all graphical elements are within reach of the user's thumb.

The second GUI 402 may further comprise a plurality of option icons. The option icons may include “medication” or “activity.” The “medication” option icon may allow the user to check his/her medication information, including a name of the medication, or a schedule of taking the medication. The “activity” option icon may allow the user to check his/her activity information, including a name of the activity, a schedule of the activity, or whether the user finishes the activity on time. The option icons may be positioned at the top of the screen, the middle of the screen, the bottom of the screen, or a location that when the user is holding the device, all graphical elements are within reach of the user's thumb.

The third GUI 403 may provide an example of a screen of information about user's activity. The third GUI 403 may comprise a plurality of graphical elements displayed on the screen. The plurality of graphical elements may be a plurality of horizontal bars arranged vertically on the screen. The color of each bar may be gray, white, blue, red, or any color. The bars may demonstrate information of the user's daily schedule. The information of the user's daily schedule may comprise when the user is scheduled to conduct an activity, whether the user finishes the activity or not, or how long it takes for the user to finish the activity. The activity may comprise having breakfast, taking different medications, having lunch, having dinner, or getting exercise. If the user does not complete an activity on time according to his/her schedule, the screen of the third GUI 403 may show different types of alerts to the user. The different types of alerts may comprise a change of colors of the bar representing the activity, or a pop-up window that reminds the user that he/she does not complete the activity.

The third GUI 403 may further comprise a plurality of option icons. The option icons may include “medication” or “activity.” The “medication” option icon may allow the user to check his/her medication information, including a name of the medication, or a schedule of taking the medication. The “activity” option icon may allow the user to check his activity information, including a name of the activity, a schedule of the activity, or whether the user finishes the activity on time. The option icons may be positioned at the top of the screen, the middle of the screen, the bottom of the screen, or a location that when the user is holding the device, all graphical elements are within reach of the user's thumb.

The fourth GUI 404 may provide an example of a screen of the dashboard. The fourth GUI 404 may comprise a plurality of graphical elements displayed on the screen. The plurality of graphical elements may be icons. The icons may demonstrate physical information of the user. The physical information of the user may comprise height of the user, weight of the user, heart rate of the user, mood of the user, or blood pressure of the user. The icons may demonstrate one or more descriptions of an exercise of the user. The descriptions of the exercise may be any type of information about the exercise, including a distance that the user has travelled during the exercise, a duration of the exercise, a pace of the exercise, steps taken during the exercise, calories burned during the exercise, power harvested during the exercise, and heart rate of the user during the exercise.

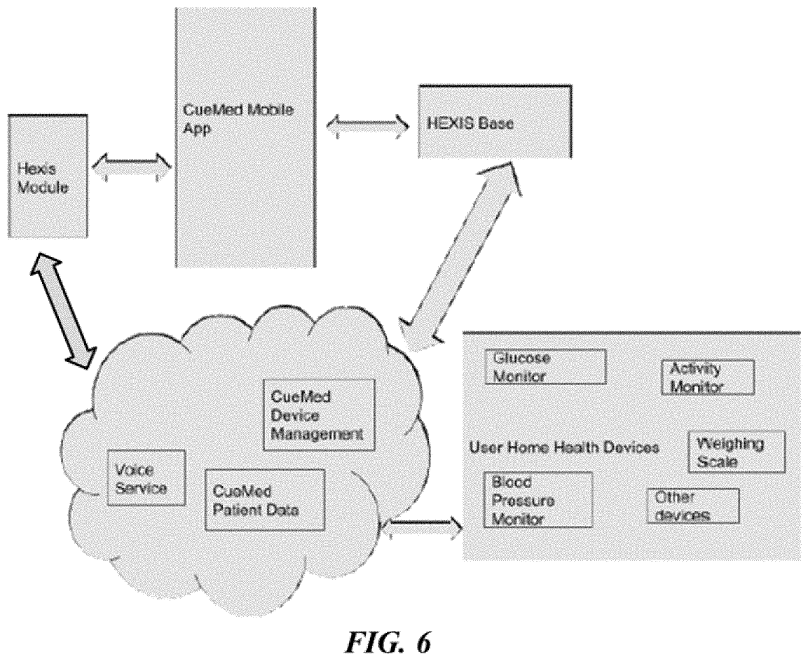

FIG. 6 shows an example schematic of communication pathways between components of a smart health management device and one or more external components. Various components of the smart health management device may be in communication with one another and in communication with a cloud computing network. For example, the base may be in communication with the cloud computing network, a mobile application (app) on the user device, and the modules. The modules may also be in communication with the mobile application, cloud computing network, and base. Additionally, mobile app may be in communication with the cloud computing network. The cloud computing network may provide device management, patient data, and voice service. The cloud computing network may be in further communication with other home health devices, such as activity monitors, blood pressure monitors, glucose monitors, weight scale, and other health devices to permit a user's health data to be stored in the cloud.

### Computer Systems

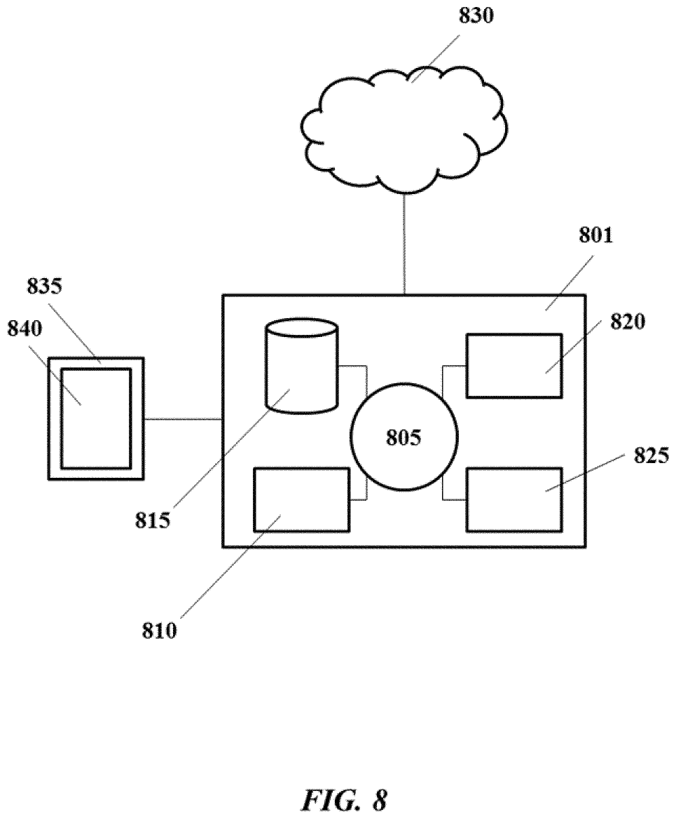

The present disclosure provides computer systems that are programmed to implement methods of the disclosure. FIG. 8 shows a computer system 801 that is programmed or otherwise configured to control a smart health management device. The computer system 801 can regulate various aspects of the smart health management device, such as, for example, changing colors of LED notification structures of the smart health management device, turning off an alert sent by the smart health management device, and switching to power saving mode of the smart health management device. The computer system 801 can be an electronic device of a user or a computer system that is remotely located with respect to the electronic device. The electronic device can be a mobile electronic device.

The computer system 801 includes a central processing unit (CPU, also “processor” and “computer processor” herein) 805, which can be a single core or multi core processor, or a plurality of processors for parallel processing. The computer system 801 also includes memory or memory location 810 (e.g., random-access memory, read-only memory, flash memory), electronic storage unit 815 (e.g., hard disk), communication interface 820 (e.g., network adapter) for communicating with one or more other systems, and peripheral devices 825, such as cache, other memory, data storage and/or electronic display adapters. The memory 810, storage unit 815, interface 820 and peripheral devices 825 are in communication with the CPU 805 through a communication bus (solid lines), such as a motherboard. The storage unit 815 can be a data storage unit (or data repository) for storing data. The computer system 801 can be operatively coupled to a computer network (“network”) 830 with the aid of the communication interface 820. The network 830 can be the Internet, an internet and/or extranet, or an intranet and/or extranet that is in communication with the Internet. The network 830 in some cases is a telecommunication and/or data network. The network 830 can include one or more computer servers, which can enable distributed computing, such as cloud computing. The network 830, in some cases with the aid of the computer system 801, can implement a peer-to-peer network, which may enable devices coupled to the computer system 801 to behave as a client or a server.

The CPU 805 can execute a sequence of machine-readable instructions, which can be embodied in a program or software. The instructions may be stored in a memory location, such as the memory 810. The instructions can be directed to the CPU 805, which can subsequently program or otherwise configure the CPU 805 to implement methods of the present disclosure. Examples of operations performed by the CPU 805 can include fetch, decode, execute, and writeback.

The CPU 805 can be part of a circuit, such as an integrated circuit. One or more other components of the system 801 can be included in the circuit. In some cases, the circuit is an application specific integrated circuit (ASIC).

The storage unit 815 can store files, such as drivers, libraries and saved programs. The storage unit 815 can store user data, e.g., user preferences and user programs. The computer system 801 in some cases can include one or more additional data storage units that are external to the computer system 801, such as located on a remote server that is in communication with the computer system 801 through an intranet or the Internet.

The computer system 801 can communicate with one or more remote computer systems through the network 830. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 801 via the network 830.

Methods as described herein can be implemented by way of machine (e.g., computer processor) executable code stored on an electronic storage location of the computer system 801, such as, for example, on the memory 810 or electronic storage unit 815. The machine executable or machine readable code can be provided in the form of software. During use, the code can be executed by the processor 805. In some cases, the code can be retrieved from the storage unit 815 and stored on the memory 810 for ready access by the processor 805. In some situations, the electronic storage unit 815 can be precluded, and machine-executable instructions are stored on memory 810.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 801, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 801 can include or be in communication with an electronic display 835 that comprises a user interface (UI) 840 for providing, for example, information about the user's activity. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 805. The algorithm can, for example, facilitate the user to control the smart health management device through the computer control systems.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1.-48. (canceled)

49. A smart health management device, comprising:
a body having a plurality of modules, wherein said plurality of modules comprise one or more compartments, wherein said one or more compartments are configured to hold medication for use by a user;
one or more proximity sensors configured to detect a presence of said user in proximity to said body;
a short-range communication interface configured to detect communication tags attached to said plurality of modules; and
one or more processors configured to (i) use said one or more proximity sensors to detect said presence of said user in proximity to said body, (ii) use said short-range communication interface to detect said communication tags, thereby detecting said plurality of modules, and (iii) upon detecting said plurality of modules, provide said user with information corresponding to said medication in said one or more compartments when said presence of said user is detected to be in proximity to said body.

50. The smart health management device of claim 49, wherein said information alerts said user to take a given medication according to a selected schedule.

51. The smart health management device of claim 49, wherein a communication tag of said communication tags provides one or more characteristics of a module of said plurality of modules.

52. The smart health management device of claim 51, wherein said one or more characteristics indicate when said module is removed from a said body and when said module is disposed in said body.

53. The smart health management device of claim 49, wherein said one or more proximity sensors is configured to detect said presence of said user when a distance between said body and said user is smaller than a predetermined distance.

54. The smart health management device of claim 49, wherein said one or more processors provides said user with said information on a mobile electronic device of said user.

55. The smart health management device of claim 54, wherein said information is provided on a user interface of said mobile electronic device of said user.

56. The smart health management device of claim 49, wherein said body further comprises a camera configured to detect a presence of medication in a compartment of said one or more compartments.

57. The smart health management device of claim 56, wherein said one or more computer processors provides said user with an alert indicating said presence of medication is detected in said compartment after a time period for said user to take said medication.

58. The smart health management device of claim 49, further comprising a sorting unit configured to sort said medication into said one or more compartments.

59. A method for providing a medication to a user, comprising:
(a) providing a smart health management device comprising (i) a body having a plurality of modules, (ii) one or more proximity sensors that detect a presence of said user in proximity to said body, and (iii) a short-range communication interface that detects communication tags attached to said plurality of modules, wherein said plurality of modules comprise one or more compartments that hold said medication for use by said user;
(b) using said one or more proximity sensors to detect a presence of said user in proximity to said body;
(c) using said short-range communication interface to detect said communication tags, thereby detecting said plurality of modules; and
(d) providing said user with information corresponding to said medication in said one or more compartments when said presence of said user is detected to be in proximity to said body.

60. The method of claim 59, wherein said information alerts said user to take a given medication according to a selected schedule.

61. The method of claim 59, wherein a communication tag of said communication tags provides one or more characteristics of said module of said plurality of modules.

62. The method of claim 61, wherein said one or more characteristics indicate when said module is removed from a said body and when said module is disposed in said body.

63. The method of claim 59, wherein said one or more proximity sensors detects said presence of said user when a distance between said body and said user is smaller than a predetermined distance.

64. The method of claim 59, wherein said information is provided to said user on a mobile electronic device of said user.

65. The method of claim 64, wherein said information is provided on a user interface of said mobile electronic device of said user.

66. The method of claim 59, wherein said body further comprises a camera that detects a presence of medication in a compartment of said one or more compartments.

67. The method of claim 66, further comprising providing said user with an alert indicating said presence of medication is detected in said compartment after a time period for said user to take said medication.

68. The method of claim 59, further comprising using a sorting unit to sort said medication into said one or more compartments.
